(19) United States
(12) Patent Application Publication
(10) Pub. No.: US 2024/0277959 A1
(43) Pub. Date: Aug. 22, 2024

(54) APPARATUS, SYSTEMS AND METHODS FOR COLLECTING ANAESTHETIC AGENTS

(71) Applicant: Class 1 Inc., Cambridge (CA)

(72) Inventors: Damian Thorne, Cambridge (CA); Mina Mehrata, Cambridge (CA); Cesar Martinez, Cambridge (CA)

(21) Appl. No.: 18/578,980

(22) PCT Filed: Jul. 12, 2022
(86) PCT No.: PCT/CA2022/051082

§ 371 Date: Jan. 12, 2024

Related U.S. Application Data
(60) Provisional application No. 63/220,802, filed on Jul. 12, 2021.

(30) Foreign Application Priority Data
Jul. 12, 2021 (CA) 3124236

(51) Int. Cl. A61M 16/00

(52) U.S. Cl. CPC A61M 16/009

(57) ABSTRACT

Systems methods for collecting an anaesthetic agent are described herein. The systems include at least one anaesthetic gas scavenging system (AGSS) for receiving exhaust gas from at least one source, the exhaust gas including the anaesthetic agent to be collected. Each AGSS comprises at least one power source for providing suction of the exhaust gas from the plurality of sources. The systems also include a collection system for recovering the anaesthetic agent from the exhaust gas. The collection system includes a compressor for compressing the exhaust gas from the AGSS to increase a pressure of the exhaust gas and at least one adsorbent tank configured to receive the compressed exhaust gas from the compressor and adsorb the anaesthetic agent from the compressed exhaust gas.

### FIELD

This disclosure relates to apparatus, systems and methods for collecting reclaiming anaesthetic agents, and in particular, to apparatus, systems and methods for reclaiming halogenated drugs from exhaust gases such as but not limited to waste air and/or exhaust gas expelled by patients in operating rooms.

### BACKGROUND

Numerous apparatuses, systems and methods are known for collecting and/or reclaiming anaesthetic agents, such as but not limited to halogenated drugs or nitrous oxide, from exhaust gases. In some of these systems, anaesthetic agents are collected from waste air and/or exhaust gas by adsorbent tanks that contain media configured to remove the anaesthetic agents.

In some instances, the exhaust gas collected from the source needs to be compressed to a high pressure (e.g., about 100 psi) for the exhaust gas to pass through the adsorbent tanks without causing a back pressure in the system. Compressing the exhaust gas to these high pressures can cause water to condense out of the exhaust gas (such as when the exhaust gas contacts the media in the adsorbent tanks), which can lead to water to collect in the adsorbent tanks and the piping therearound, causing either inefficient adsorption of the anaesthetic agents or oxidation of the piping. Further, in some instances, compressing the exhaust gases to these pressures can lead to the formation of hydrofluoric acid (HF) in the collection tanks and non-medical grade piping.

Accordingly, there is a need for new apparatus, systems and methods for collecting reclaiming anaesthetic agents.

### SUMMARY

In accordance with a broad aspect, a system for collecting an anaesthetic agent is described herein. The system includes at least one anaesthetic gas scavenging system (AGSS) for receiving exhaust gas from at least one source, the exhaust gas including the anaesthetic agent to be collected. Each AGSS includes at least one power source for providing suction of the exhaust gas from the plurality of sources. The system also includes a collection system for recovering the anaesthetic agent from the exhaust gas. The collection system includes a compressor configured to receive the exhaust gas from the AGSS, increase a pressure of the exhaust gas and emit a compressed exhaust gas. The collection system also includes at least one adsorbent tank configured to receive the compressed exhaust gas from the compressor and adsorb the anaesthetic agent from the compressed exhaust gas.

In at least one embodiment, the compressor is configured to emit the compressed exhaust gas at a pressure that inhibits condensation of water vapor in the at least one adsorbent tank.

In at least one embodiment, the pressure of the exhaust gas at an inlet of the compressor is less than 1 psi.

In at least one embodiment, the compressed exhaust gas has a pressure in a range of about 5 psi to about 15 psi at an outlet of the compressor.

In at least one embodiment, inhibiting the condensation of water vapor in the at least one adsorbent tank increases an exchange interval of the at least one adsorbent tank.

In at least one embodiment the system further comprises a controller configured to: receive pressure data from a pressure sensor positioned downstream from the compressor; and when the pressure data exceeds a threshold pressure value, direct the compressor to decrease the pressure of the compressed exhaust gas.

In at least one embodiment, the pressure sensor is positioned between the compressor and the at least one adsorbent tank.

In at least one embodiment, the pressure sensor is positioned downstream from the at least one adsorbent tank.

In at least one embodiment, the controller is further configured to: receive temperature data from a temperature sensor positioned downstream from the compressor; and when the temperature data exceeds a threshold temperature value, direct the compressor to decrease the pressure of the compressed exhaust gas.

In at least one embodiment, the temperature sensor is positioned between the compressor and the at least one adsorbent tank.

In at least one embodiment the system further comprises a monitoring system configured to monitor the controller and/or the collection system.

In at least one embodiment, the monitoring system is configured to remotely monitor the controller and/or the collection system.

In at least one embodiment, the at least one adsorbent tank is a multi-stage adsorption tank.

In accordance with a broad aspect, a method of collecting an anaesthetic agent is described herein. The method includes receiving, at a compressor of a collection system, an exhaust gas from a source, the exhaust gas comprising the anaesthetic agent to be collected; compressing the exhaust gas, by the compressor, to increase a pressure of the exhaust gas and output a compressed exhaust gas; and collecting the anaesthetic agent from the compressed exhaust gas in at least one adsorbent tank, the at least one adsorbent tank being configured to adsorb the anaesthetic agent from the compressed exhaust gas.

In at least one embodiment, the method further comprises controlling, by a controller, one or more parameters of the compressed exhaust gas in response to feedback data received at the controller, the controller being configured to receive the feedback data and transmit one or more feedback signals to one or more components of the collection system to control the one or more parameters of the compressed exhaust gas and to inhibit condensation of the compressed exhaust gas in the at least one adsorbent tank.

In at least one embodiment of the method, the one or more parameters of the compressed gas is a temperature of the compressed gas at a position downstream from the compressor; the feedback data is temperature data; and when the temperature data exceeds a threshold temperature value, the method includes directing, but the controller, the compressor to decrease a pressure of the compressed exhaust gas at an outlet of the compressor.

In at least one embodiment, the one or more parameters of the compressed gas is the temperature of the compressed gas at a position downstream from the compressor and downstream from the at least one adsorbent tank.

In at least one embodiment of the method, the one or more parameters of the compressed gas is a pressure of the compressed gas at a position downstream from the compressor; the feedback data is pressure data; and when the pressure data exceeds a threshold pressure value, the method includes directing, but the controller, the compressor to decrease a pressure of the compressed exhaust gas at an outlet of the compressor.

In at least one embodiment, the one or more parameters of the compressed gas is the pressure of the compressed gas at a position downstream from the compressor and downstream from the at least one adsorbent tank.

In at least one embodiment of the method, the compressed exhaust gas has a pressure in a range of about 5 psi to about 15 psi.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a schematic diagram of a system for collecting anaesthetic gases, according to at least one embodiment described herein.

FIG. 2 is a schematic diagram of a central collection system of the system for collecting anaesthetic gases of FIG. 1, according to at least one embodiment described herein.

FIG. 3 is a flow diagram of a method of monitoring collection of an anaesthetic agent from an exhaust gas, according to at least one embodiment described herein.

Further aspects and features of the example embodiments described herein will appear from the following description taken together with the accompanying drawings.

### DESCRIPTION OF EXAMPLE EMBODIMENTS

Various apparatuses, methods and compositions are described below to provide an example of at least one embodiment of the claimed subject matter. No embodiment described below limits any claimed subject matter and any claimed subject matter may cover apparatuses and methods that differ from those described below. The claimed subject matter are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed subject matter. Any subject matter that is disclosed in an apparatus, method or composition described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term, such as 1%, 2%, 5%, or 10%, for example, if this deviation does not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

It should also be noted that, as used herein, the wording “and/or” is intended to represent an inclusive—or. That is, “X and/or Y” is intended to mean X, Y or X and Y, for example. As a further example, “X, Y, and/or Z” is intended to mean X or Y or Z or any combination thereof. Also, the expression of A, B and C means various combinations including A; B; C; A and B; A and C; B and C; or A, B and C.

The following description is not intended to limit or define any claimed or as yet unclaimed subject matter. Subject matter that may be claimed may reside in any combination or sub-combination of the elements or process steps disclosed in any part of this document including its claims and figures. Accordingly, it will be appreciated by a person skilled in the art that an apparatus, system or method disclosed in accordance with the teachings herein may embody any one or more of the features contained herein and that the features may be used in any particular combination or sub-combination that is physically feasible and realizable for its intended purpose.

Recently, there has been a growing interest in developing new apparatus, systems and methods of collection anaesthetic gases.

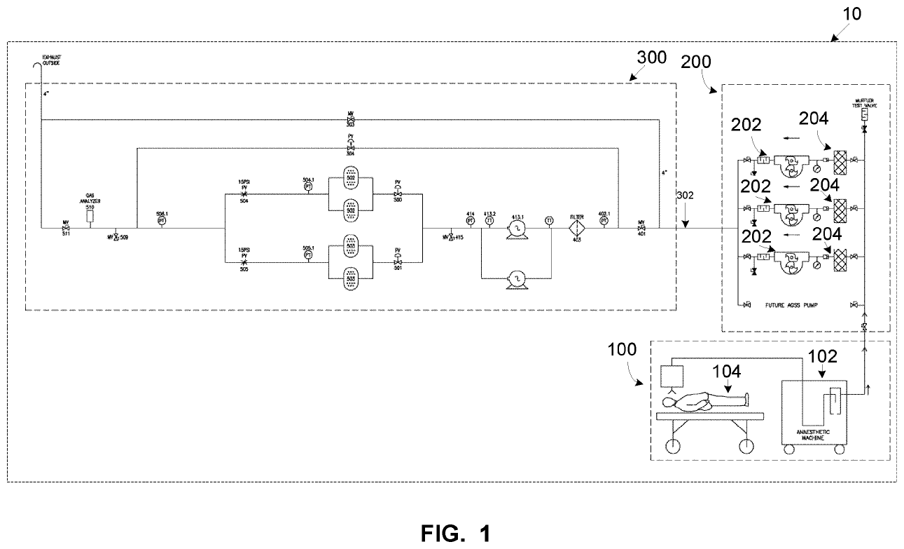

Referring to FIG. 1, illustrated therein is a system 10 for collecting anaesthetic agents from exhaust gases. System 10 includes one or more sources of the exhaust gas 100 and a collection system 300.

In FIG. 1, the one or more sources of the exhaust gas 100 are represented by the collection of units/spaces within the dashed box identified with the reference number 100. In at least one embodiment, the one or more sources of exhaust gas 100 may be from one or more operating rooms in a healthcare facility. For example, in at least one embodiment, such as the embodiment shown in FIG. 1, the one or more operating rooms 100 may have an anaesthetic machine 102 connected to one or more patients 104 for administering one or more anaesthetic agents (such as but not limited to halogenated drugs, nitrous oxide, etc.) during or in association with a medical procedure. In some cases, the anaesthetic machine 102 may also collect exhaust gases from the patient 104 and direct those exhaust gases to the collection system 300, for example, through an exhaust port in the operating room 100. In some embodiments, a conservation valve (not shown) may be located between the anaesthetic machine 102 and the exhaust port.

In some embodiments, the exhaust gases may come from other sources, such as but not limited to patient rooms in a hospital, an outpatient clinic, a surgery clinic, a doctor's office, an oral surgery clinic, a veterinary clinic, or other types of healthcare facilities.

FIG. 1 also shows an advanced gas scavenging system (AGSS) 200 fluidly coupled to the one or more sources of the exhaust gas 100 and to collection system 300. AGSS 200 is represented in FIG. 1 by the collection of units within the dashed box identified with the reference number 200. As shown in FIG. 1, AGSS 200, also sometimes referred to as a Waste Anaesthetic Gas Disposal (“WAGD”) system, may be connected to the one or more sources of exhaust gas 100 and to the collection system 300. As shown in FIG. 1, the AGSS 200 may be located between the one or more sources of exhaust gas 100 and the collection system 300.

The AGSS 200 draws exhaust gases from the one or more sources of exhaust gas 100 and directs these exhaust gases to the collection system 300. For example, the AGSS 200 may include one or more power source(s) 202, such as but not limited to a vacuum pump, or a blower, or a fan or the like, connected to the exhaust port(s) of the one or more sources of exhaust gas 100 through piping, ducting or other mediums for transporting liquids or gases (collectively referred to as “piping” herein). In one or more embodiments, the power source(s) 200 may be connected to an inlet port of the collection system 300 through piping. In the embodiment shown in FIG. 1, the AGSS 200 includes, but is not limited to, three power sources 202 operating in series.

AGSS 200 may also include one or more filters 204, such as but not limited to a high-efficiency particulate air (HEPA) filter or an ultra-low particulate air (ULPA) filter, or the like. Filter 204 may provide for removing impurities such as but not limited to dust, dirt, etc. present in the exhaust gas. Filters 204 are positioned between the power source 202 and the exhaust port of the one or more sources of gas 100 to remove any impurities from the exhaust gas before the exhaust gas enters the power source 202. In the embodiment shown in FIG. 1, AGSS 200 includes three filters 204, positioned immediately before one of the three power sources 202 to remove any impurities from the exhaust gas before the exhaust gas enters the power source 202.

In some embodiments, the AGSS 200 may include two or more power sources 202 connected in parallel. Having an additional power source 202 may provide a back-up in case one power source 202 stops operating (e.g. breaks down or needs maintenance), which may improve system redundancy. Additional power sources 202 may also increase the suction, for example, when the AGSS 200 is connected to larger systems (e.g. a greater number of operating rooms). Furthermore, in some embodiments there may be more than one AGSS 200, which may be connected in parallel, for example, to provide redundancy.

In at least one embodiment, AGSS 200 includes variable frequency drive (VFD) to maintain an exhaust pressure (i.e., a pressure of inlet stream 302 as shown in FIG. 1) less than about 1 psi.

Although FIG. 1 shows an AGSS 200 fluidly coupled to both sources of gas 100 and collection system 300, it should be understood that other subsystems may be fluidly coupled to the sources of gas 100 and collection system 300. For example, a medical vacuum system may be fluidly coupled to the sources of gas 100 and collection system 300. Typically, medical vacuum systems are fluidly coupled to patient rooms in a health facility such as a hospital, whereas AGSSs are typically fluidly coupled to operating rooms in a health facility such as a hospital.

System 10 also includes a collection system 300 for collecting anesthetic agents, such as but not limited to halogenated drugs, for later reclaiming, from the one or more sources of exhaust gas 100, according to at least one embodiment. Collection system 300 may be installed in a healthcare facility such as a hospital and may be centrally located such that it is in fluid communication with one or more sources of exhaust gas 100.

In at least one embodiment, the collection system 300 may be remotely located relative to the one or more sources of exhaust gas 100. For example, collection system 300 may be located within the hospital at a location that is both central and remote relative to one or more sources of exhaust gas 100 while remaining in fluid communication with the one or more sources of exhaust gas 100 via piping.

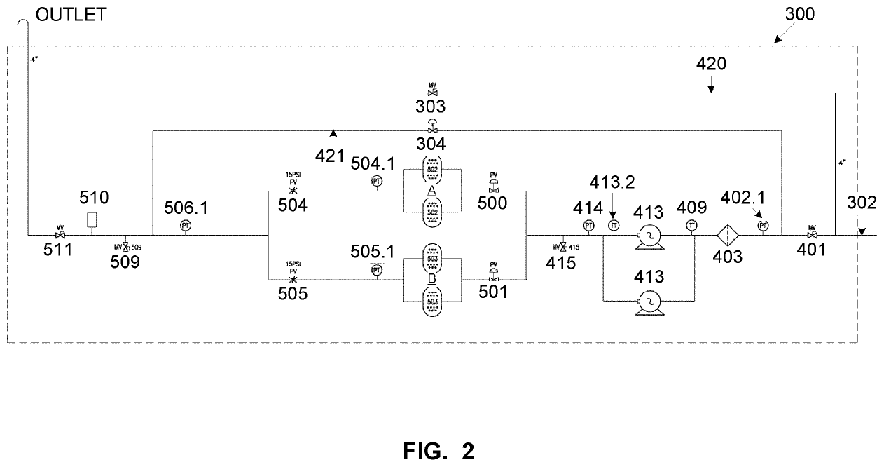

Collection system 300, shown in FIG. 2, includes an inlet stream 302, at least one compressor 413, and at least one adsorption tank 502. In the embodiment shown in FIG. 2, the collection system 300 also includes a second adsorption tank 503 and a gas analyzer 510.

Under normal operating conditions, an exhaust gas entering collection system 300 from the AGSS 200 enters compressor 413 configured to increase the pressure of the exhaust gas. Compressor 413 receives the exhaust gas at a low pressure (e.g. under about 1 psi) and emits or outputs a compressed exhaust gas having a higher pressure (e.g. a pressure in a range of about 5 psi to about 15 psi, or in a range of about 8 psi to about 12 psi, or of about 15 psi).

In previous systems, the exhaust gas entering previous collections systems has been compressed to much higher pressures than is described herein. For example, in some systems, the exhaust gas entering previous collections systems has been compressed to pressures of about 100 psi. Herein, the lower pressure of the gas exiting the compressor 413 may offer a number of advantages, such as but not limited to lower energy requirements (e.g. lower energy required by the compressor and/or the collection system 300). In at least one embodiment, the lower pressure of the gas exiting the compressor may provide for the present system to be about 37.5% more energy efficient relative to similar prior art systems. Further, the lower pressure of the gas exiting the compressor 413 relative to previous systems may offer other advantages, such as but not limited to safety advantages over similar previous systems. For example, in at least one embodiment, the lower pressure of the gas exiting the compressor 413 relative to previous systems may provide for the downstream adsorbent tanks to not require a Canada Registration Number (CRN) certificate for pressurized vessels.

As noted above, compressor 413 is configured to increase the pressure of the exhaust gas and emit a compressed exhaust gas. Compressor 413 may be a variable speed compressor or another type of compressor such as a fixed speed compressor. In at least one embodiment, compressor 413 may be an oilless compressor, such as but not limited to an oil-free rotary screw compressor, an oil-free rotary lobe compressor, an oil-free rotary scroll compressor, an oil-free reciprocating compressor, or the like. Use of an oilless compressor as compressor 413 may promote purity of the compressed exhaust gas emitted from the compressor 413 by inhibiting a presence of oil and/or other impurities in compressed exhaust gas. This may be important for improving an efficiency of adsorbing the anaesthetic agent in the downstream adsorbent tanks 502 and 503.

In at least one embodiment, a flow rate through the compressor 413 may be about 2600 liters per minute and the pressure may be about 15 psi.

In at least one embodiment, compressor 413 provides for the pressure of the exhaust gas to be increased from the outlet of the AGSS, while minimizing the presence of water due to condensation in the downstream adsorbent tanks 502 and 503. As noted above, as the exhaust gas containing the anaesthetic agent typically comes from a patient and/or an operating room in a health facility, the exhaust gas typically has a high humidity (e.g. in a range of about 80% to 100% relative humidity). In prior art systems for removing anesthetic agents from exhaust gases, condensation of water vapor from the exhaust gas in and/or around the adsorbent tank(s) 502 and 503 has been an issue that decreased efficiency of adsorption of the anaesthetic gas in the adsorbent tank(s) 502 and 503. For example, in prior art systems, condensation of water vapor from the exhaust gas in and/or around the adsorbent tank(s) resulted in the adsorbent tank(s) having to be exchanged before reaching a peak absorbing capacity because of the presence of water in the adsorbent tank.

Previously, one or more dryers were required to be positioned before any adsorbent tank to remove water from the exhaust gas and inhibit the formation of water by condensation in/or around the adsorbent tanks. For instance, refrigerant dryers have previously been used to reduce water content of the exhaust gas received from the AGSS 200. Refrigerant dryers provided for moisture removal from the exhaust gas because the flow rate of the exhaust gas through the dryer could remain high. Refrigerant dryers require at least 50 psi to operate efficiently as a dryer.

For the collection systems described herein, such as collection system 300, the exhaust gas received at compressor 413 from the AGSS 200 has a much lower pressure than in previous systems (e.g. less than about 10 psi, or less than about 5 psi, or about 1 psi) and the pressure of the compressed exhaust gas emitted from the compressor 413 is lower than in previous systems, which inhibits potential production of HF downstream of compressor 413 and/or inhibits water condensation downstream from the compressor 413 (e.g. in the adsorbent tanks(s)). Inhibiting condensation of water vapor downstream of compressor 413 may provide for increasing an exchange interval (i.e. a period of time during which the adsorbent tank adsorbs anaesthetic agent before it can no longer absorb an anaesthetic agent and therefore needs to be exchanged) of the at least one adsorbent tank.

Further, in previous systems, media inside of the adsorbent tank(s) restricted flow of the exhaust gas through the tank(s), thereby providing for a back pressure on the AGSS pumps. In the collections systems described here, such as collection system 300, presence of compressor 413 inhibits a back pressure on the AGSS pumps.

As noted above, compressor 413 receives the exhaust gas at a low pressure (e.g. under about 1 psi) and emits or outputs a compressed exhaust gas having a higher pressure (e.g. a pressure in a range of about 5 psi to about 15 psi, or in a range of about 8 psi to about 12 psi, or of about 15 psi). In at least one embodiment, compressor 413

In at least one embodiment, more than one compressor 413 may be provided in the collection system 300. For instance, in the embodiment of collection system 300 shown in FIG. 2, two compressors 413 are shown operating in series. In this case, two compressors 413 provide the collection system 300 with redundancy in the event that one compressor 413 is inoperable.

During normal operation, the compressed exhaust gas stream exiting the compressor 413 passes through one or more adsorbent tanks 502 and 503 configured to remove all halogenated agents from the compressed exhaust gas. It should be understood that the compressed exhaust gas stream may pass through the one or more adsorbent tanks 502 and 503 even when they are saturated with anaesthetic agent and cannot remove anaesthetic agents from the exhaust gas flow. However, once one of the adsorbent tanks is full, for example adsorbent tank 502, the system is configured for the compressed exhaust gas stream to flow into the other adsorbent tank, for example adsorbent tank 503, while adsorbent tank 502 is replaced with a new tank with fresh adsorbent. The full adsorbent tank 502 can then be transferred to a desorbing site where the anaesthetic agents present in the full adsorbent tank 502 may be for later reuse.

In at least one embodiment, as the compressed exhaust gas passes through each of the one or more adsorbent tanks 502 and 503, the compressed exhaust gas passes through a bed of adsorbent material (e.g. media) until the adsorbent material in the one or more of the adsorbent tanks 502 and 503 is saturated to the extent that breakthrough of the anaesthetic agents is determined (e.g. halogenated hydrocarbons are detected at the outlet of the one or more of the adsorbent tanks 502 and 503).

In some embodiments, after recovering the anaesthetic agents from one of the adsorbent tanks 502 and 503 (e.g. after saturation and exchange for new tank) at the desorbing site, adsorbent tank 502 may be reinstalled in the collection system 300. Accordingly, each adsorbent tank 502 may be reused multiple times.

Water in the adsorbent tanks 502 and 503 inhibits desorption of the anaesthetic agents from the adsorbent material. In at least one embodiment, compressor 413 providing the compressed exhaust gas to the adsorbent tanks 502, 503 at a much lower pressure than in previous systems (e.g. a pressure in a range of about 5 psi to about 15 psi, or in a range of about 8 psi to about 12 psi, or of about 15 psi), thereby reducing condensation in the adsorbent tanks 502, 503, may increase an efficiency of desorption of the anaesthetic agent(s) in the adsorbent tanks 502 and 503 after the adsorbent tanks 502 and 503 have been removed from collection system 300 relative to prior art systems. For instance, by inhibiting condensation of water vapor in the adsorbent tanks 502 and 503, desorption of the anaesthetic agent(s) from the adsorbent tanks 502 and 503 may be more efficient (e.g. take less time, have a higher removal effectiveness, require less energy, etc.) than in prior art systems where water is present in the adsorbent tanks 502 and 503. Similarly, water in the adsorbent tanks 502 and 503 inhibits conversion of desorbed anesthetic agents to other chemicals, desorption of the anaesthetic agents from the adsorbent material. Compressor 413 providing the compressed exhaust gas to the adsorbent tanks 502, 503 at a much lower pressure than in previous systems (e.g. a pressure in a range of about 5 psi to about 15 psi, or in a range of about 8 psi to about 12 psi, or of about 15 psi), thereby reducing condensation in the adsorbent tanks 502, 503, may also increase an efficiency of converting the desorbed anaesthetic agent(s) to other chemicals relative to prior art systems.

When recovering anaesthetic agents from the one or more adsorbent tanks 502 and 503, it may be desirable to keep the collection system 300 operational so as to provide continuous collection of anaesthetic agents. Accordingly, the collection system 300 may include a plurality of adsorbent tanks (e.g. collector bank “A” and collector bank “B”, as shown in FIG. 2) connected together in parallel so that at least one adsorbent tank remains operational while one or more other adsorbent tanks are being processed to remove the captured anaesthetic agents, for example. In at least one embodiment, the adsorbent tanks 502 and 503 each have a diameter of about 18 inches and a volume of about 11,000 cubic inches. In at least one embodiment, each of the adsorbent tanks 502 and 503 has a flow rate of about 300 liters per minute.

When there is a plurality of adsorbent tanks 502, 503 connected in parallel, the inlet of each of adsorbent tanks 502, 503 may include a valve 500, such as is shown in FIG. 2 for adsorbent tank 502 (or valve 501 such as is shown in FIG. 2 for adsorbent tank 503) for selectively opening and closing the flow of compressed exhaust gases into the respective adsorbent tanks 502 and 503 from the compressor 413. Valves 50 or 51 may be closed when removing the respective adsorbent tanks 502 or 503 from the system 300 and the other of valves 50 or 51 may be open such that the compressed exhaust gas from compressor 413 is permitted to flow into those other adsorbent tanks. Accordingly, the collection system 300 may remain operational while some of the adsorbent tanks are being processed.

Collection system 300 may also include an analyzer 510 for measuring an amount of anaesthetic agent collected and for detecting when one of the adsorbent tanks 502 and 503 reaches break through, for example, so that anaesthetic agents can be reclaimed, by removing/exchanging the adsorbent tanks 502 or 503 (depending which tank reaches break through) from the collection system 300. In some embodiments, the analyzer 510 may measure a presence of an anaesthetic agent in the piping downstream from the one or more adsorbent tanks 502 and 503. Upon exceeding a threshold limit, the analyzer 510 may provide a signal to a control system (described further, below) to control the valves 500 and 501 to switch over the flow of the compressed exhaust gas into one or more of the adsorbent tanks 502 and 503 so that one or more of the adsorbent tanks 502 and 503 can be removed/exchanged from the system 300.

As noted above, collection system 300 may also include a controller (not shown) for controlling various subcomponents of the collection system 300. In some embodiments, the controller (such as a control panel) may be configured to automate system 300. For example, the controller may monitor the status of the adsorbent tanks 502 and 503 (e.g. via a sensor therein) to determine if they are full and need to be replaced or regenerated.

In another example, as noted above, the controller may be communicatively coupled to the analyzer 510 and the valves 500, 501 and be configured to receive a signal from the analyzer 510 to close one or more of the valves 500, 501 and open one or more of the valves 500, 501 in response to the analyzer detecting the presence of one or more anesthetic agents downstream from the one or more adsorbent tanks 502 and 503 in an amount that exceeds a threshold limit.

In another example, the controller may be communicatively coupled to the one or more compressors 413 and to one or more sensors positioned downstream from the one or more compressors 413, such as but not limited to a temperature sensor 413.2 and/or a pressure sensor 414. In at least one embodiment, the controller is configured to receive data from the temperature sensor 413.2 and/or pressure sensor 414 and transmit a signal to each of the one or more compressors 413 to decrease the pressure of the exhaust gas exiting the scavenging system 200. For instance, the controller may be configured to receive data from the temperature sensor 413.2 and/or pressure sensor 414 indicating a likelihood of compressor 413 malfunctioning. In another embodiment, the controller may be communicatively coupled to one or more valves, such as but not limited to bypass valve 304 and may be configured to send a signal to the one or more valves 500 and 501 to open or close in response to sensing back pressure in gas scavenging system 200 (e.g. via pressure sensor 414 and/or pressure sensor 402.1).

In some embodiments, the collection system 300 may include a bypass or failsafe stream 420 so that exhaust gas received into system 300 from AGSS 200 may be directed to bypass compressor 413 and adsorbent tanks 502 and 503 and/or other components of the collection system 300. It may be desirable to use bypass stream 420 when conducting maintenance or servicing of the system 300. To direct the exhaust gas into the bypass stream 420, valve 401 is closed.

In another embodiment a second bypass stream 421 is provided in the example embodiment shown in FIG. 2. Exhaust gas from AGSS 200 can be directed into the second bypass stream 421 by bypass valve 304 connected downstream of the inlet port of the collection system 300. In some embodiments, the bypass valve 304 may be operated automatically. In this sense, the bypass valve 304 may be a fail-safe valve that enhances safety, such as a normally open, energized closed, spring return ball valve, piston valve, blow-off valve or a burst disc. In other embodiments, the bypass valve 303 may be operated manually.

As described above, the collection system 300 may be located centrally and remotely from one or more sources of exhaust gas 100 (e.g. the operating rooms). Having a centrally located system may decrease the overall cost of the collecting anaesthetic agents in comparison to conventional systems that are—in most operating rooms. As such, the initial capital costs of providing a large central collection system may be less than the accumulative cost of several smaller localized systems.

Furthermore, operational costs of a centralized system may be lower in comparison to a local system because the collection system 300 may be able to more easily implement a monitoring system that controls the collection and reclamation of anaesthetic agents from each source of exhaust gas. Tracking the collection from a centralized system makes determining when servicing or maintenance is required easier and logistically cheaper. For example, the monitoring system may track the capacity of the adsorbent tanks 502 and 503 so that they may be replaced or regenerated once they are saturated or otherwise full. Monitoring one central system in this way may be less expensive than monitoring several individual localized systems. In at least one embodiment, the monitoring system may be a remote (i.e. situated away from the collection system) monitoring system and may be configured to remotely monitor more than one collection system at a single time. For example, in at least one embodiment, the remote monitoring system may be configured to monitor and/or measure a volume of anaesthetic collected.

In at least one embodiment, collection system 300 may also include a filter 403 for filtering out impurities from the exhaust gas received from the AGSS 200. Filter 403 is positioned upstream from the compressor 413 to inhibit impurities from entering the compressor 413.

In at least one embodiment, collection system 300 may also include one or more temperature sensors 406 positioned upstream of the compressor 413 to monitor a temperature of the exhaust gas received from the AGSS 200.

In at least one embodiment, collection system 300 may include a valve 415 for controlling the flow of the compressed exhaust gas from the compressor 413 towards the adsorbent tanks 502 and 503.

In at least one embodiment, collection system 300 may also include one or more temperature sensors 406 positioned upstream of the compressor 413 to monitor a temperature of the exhaust gas received from the AGSS 200.

In at least one embodiment, collection system 300 may also include one or more pressure sensors 504.1 and 505.1 positioned downstream of the adsorbent tanks 502 and 503 to monitor the pressure of the gas stream(s) output from the adsorbent tanks 502 and 503.

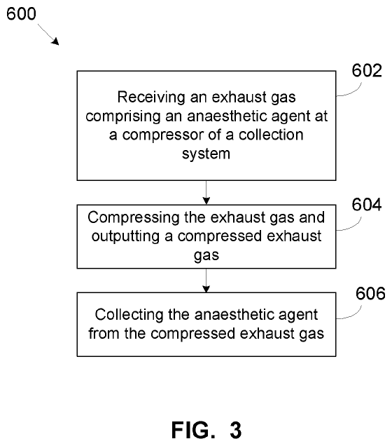

Referring now to FIG. 3, illustrated therein is a flow diagram of steps of a method 600 of collecting an anaesthetic agent.

At a first step 602, an exhaust gas is received at a collection system 300 from a source, for example via an AGSS 200. The exhaust gas includes the anaesthetic agent to be collected. As noted above, the AGSS 200 typically includes at least one power source for providing suction to draw the exhaust gas from the source and direct the exhaust gas towards the collection system 300. Specifically, at first step 602, the exhaust gas is received at a compressor 413 of collection system 300. As noted above, the compressor 413 is configured to compress the exhaust gas to increase a pressure of the exhaust gas and output a compressed exhaust gas.

At a second step 604, the received exhaust gas is compressed by the compressor 413 and a compressed exhaust gas is output from the compressor 413.

In a third step 606, the method 600 further includes collecting the anaesthetic agent from the compressed exhaust gas having an increased pressure in at least one adsorbent tank, the at least one adsorbent tank being configured to adsorb the anaesthetic agent from the compressed exhaust gas.

Method 600 may optionally include controlling, by a controller, one or more parameters of the compressed exhaust gas in response to feedback data received at the controller, the controller being configured to receive the feedback data and transmit one or more feedback signals to one or more components of the collection system to control the one or more parameters of the compressed exhaust gas to inhibit condensation of the compressed exhaust gas in the at least one adsorbent tank. For example, the controller may be configured to, for example, receive pressure data and/or temperature data as the feedback data from one or more pressure and/or temperature sensors positioned downstream from the compressor 413. The controller may also be configured to transmit one or more feedback signals to one or more valves to control the flow of the exhaust gas and/or the compressed exhaust gas within the collection system 300.

While some of the embodiments described above may refer to collecting or reclaiming halogenated drugs, the apparatus, systems and methods described herein may be used for the collection of several types of anaesthetic agents, including halogenated drugs and other agents.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A system for collecting an anaesthetic agent, the system comprising:
at least one anaesthetic gas scavenging system (AGSS) for receiving exhaust gas from at least one source, the exhaust gas including the anaesthetic agent to be collected, each AGSS comprising at least one power source for providing suction of the exhaust gas from the plurality of sources; and
a collection system for recovering the anaesthetic agent from the exhaust gas, the collection system comprising:
a compressor configured to receive the exhaust gas from the AGSS, increase a pressure of the exhaust gas and emit a compressed exhaust gas; and
at least one adsorbent tank configured to receive the compressed exhaust gas from the compressor and adsorb the anaesthetic agent from the compressed exhaust gas.

2. The system of claim 1, wherein the compressor is configured to emit the compressed exhaust gas at a pressure that inhibits condensation of water vapor in the at least one adsorbent tank.

3. The system of claim 1, wherein the pressure of the exhaust gas at an inlet of the compressor is less than 1 psi.

4. The system of claim 1, wherein the compressed exhaust gas has a pressure in a range of about 5 psi to about 15 psi at an outlet of the compressor.

5. The system of claim 2, wherein inhibiting the condensation of water vapor in the at least one adsorbent tank increases an exchange interval of the at least one adsorbent tank.

6. The system of claim 1 further comprising a controller configured to:
receive pressure data from a pressure sensor positioned downstream from the compressor; and
when the pressure data exceeds a threshold pressure value, direct the compressor to decrease the pressure of the compressed exhaust gas.

7. The system of claim 6, wherein the pressure sensor is positioned between the compressor and the at least one adsorbent tank.

8. The system of claim 6, wherein the pressure sensor is positioned downstream from the at least one adsorbent tank.

9. The system of claim 6, wherein the controller is further configured to:
receive temperature data from a temperature sensor positioned downstream from the compressor; and
when the temperature data exceeds a threshold temperature value, direct the compressor to decrease the pressure of the compressed exhaust gas.

10. The system of claim 9, wherein the temperature sensor is positioned between the compressor and the at least one adsorbent tank.

11. The system of claim 6 further comprising a monitoring system configured to monitor the controller and/or the collection system.

12. The system of claim 11, wherein the monitoring system is configured to remotely monitor the controller and/or the collection system.

13. The system of claim 1, wherein the at least one adsorbent tank is a multi-stage adsorption tank.

14. A method of collecting an anaesthetic agent, the method comprising:
receiving, at a compressor of a collection system, an exhaust gas from a source, the exhaust gas comprising the anaesthetic agent to be collected;
compressing the exhaust gas, by the compressor, to increase a pressure of the exhaust gas and output a compressed exhaust gas; and
collecting the anaesthetic agent from the compressed exhaust gas in at least one adsorbent tank, the at least one adsorbent tank being configured to adsorb the anaesthetic agent from the compressed exhaust gas.

15. The method of claim 14, further comprising controlling, by a controller, one or more parameters of the compressed exhaust gas in response to feedback data received at the controller, the controller being configured to receive the feedback data and transmit one or more feedback signals to one or more components of the collection system to control the one or more parameters of the compressed exhaust gas and to inhibit condensation of the compressed exhaust gas in the at least one adsorbent tank.

16. The method of claim 15, wherein:
the one or more parameters of the compressed gas is a temperature of the compressed gas at a position downstream from the compressor;
the feedback data is temperature data; and
when the temperature data exceeds a threshold temperature value, the controller directs the compressor to decrease a pressure of the compressed exhaust gas at an outlet of the compressor.

17. The method of claim 16, wherein the one or more parameters of the compressed gas is the temperature of the compressed gas at a position downstream from the compressor and downstream from the at least one adsorbent tank.

18. The method of claim 15, wherein:
the one or more parameters of the compressed gas is a pressure of the compressed gas at a position downstream from the compressor;
the feedback data is pressure data; and
when the pressure data exceeds a threshold pressure value, the controller directs the compressor to decrease a pressure of the compressed exhaust gas at an outlet of the compressor.

19. The method of claim 18, wherein the one or more parameters of the compressed gas is the pressure of the compressed gas at a position downstream from the compressor and downstream from the at least one adsorbent tank.

20. The method of claim 14, wherein the compressed exhaust gas has a pressure in a range of about 5 psi to about 15 psi.
